(19) United States
(12) Patent Application Publication
(10) Pub. No.: US 2025/0001179 A1
(43) Pub. Date: Jan. 2, 2025

(54) FEEDBACK CONTROL OF NEUROMODULATION

(71) Applicant: Saluda Medical Pty Ltd, Macquarie Park (AU)

(72) Inventor: James Hamilton Wah, Artarmon (AU)

(21) Appl. No.: 18/883,401

(22) Filed: Sep. 12, 2024

Related U.S. Application Data
(63) Continuation of application No. 17/454,237, filed on Nov. 9, 2021, now Pat. No. 12,138,457.
Application No. 17/454,237 is a continuation of application No. 16/091,428, filed on Oct. 4, 2018, now Pat. No. 11,191,966.

(30) Foreign Application Priority Data
Apr. 5, 2016 (AU) 2016901264

(51) Int. Cl. A61N 1/36; A61B 5/00; A61B 5/388; A61N 1/05; A61N 1/18; A61N 1/372; A61N 1/378

(52) U.S. Cl. CPC A61N 1/36132; A61B 5/0031; A61B 5/388; A61B 5/4836; A61N 1/0551; A61N 1/18; A61N 1/36067; A61N 1/36071; A61N 1/36075; A61N 1/36139; A61N 1/37264; A61N 1/3787

(57) ABSTRACT

An automated method of controlling neural stimulation. A neural stimulus is applied to a neural pathway in order to give rise to an evoked action potential on the neural pathway, and the stimulus is defined by at least one stimulus parameter. A neural compound action potential response evoked by the stimulus is measured. From the measured evoked response a feedback variable such as observed ECAP voltage (V) is derived. A feedback loop is completed by using the feedback variable to control the at least one stimulus parameter value for a future stimulus. The method adaptively compensates for changes in a gain of the feedback loop caused by electrode movement relative to the neural pathway. A compensating transfer function is applied to the feedback variable, the compensating transfer function being configured to compensate for both (i) a distance-dependent transfer function of stimulation, and (ii) a distance dependent transfer function of measurement which is distinct from (i).

### CROSS REFERENCE TO RELATED APPLICATIONS

This application is a continuation of U.S. application Ser. No. 17/454,237, filed Nov. 9, 2021, which is a continuation of U.S. application Ser. No. 16/091,428, filed Oct. 4, 2018 and issued on Dec. 7, 2021 as U.S. Pat. No. 11,191,966, which is a 371 national stage of International Application No. PCT/AU2017/050296, filed Apr. 5, 2017, which claims the benefit of Australian Application No. 2016901264, filed Apr. 5, 2016. All of the aforementioned applications are incorporated in their entirety herein by reference.

### TECHNICAL FIELD

The present invention relates to controlling a neural response to a stimulus, and in particular relates to measurement of a compound action potential by using one or more electrodes implanted proximal to the neural pathway, in order to provide feedback to control subsequently applied stimuli.

### BACKGROUND OF THE INVENTION

There are a range of situations in which it is desirable to apply neural stimuli in order to give rise to an evoked compound action potential (ECAP). For example, neuromodulation is used to treat a variety of disorders including chronic neuropathic pain, Parkinson's disease, and migraine. A neuromodulation system applies an electrical pulse to neural tissue in order to generate a therapeutic effect.

When used to relieve neuropathic pain originating in the trunk and limbs, the electrical pulse is applied to the dorsal column (DC) of the spinal cord, referred to as spinal cord stimulation (SCS). Such a system typically comprises an implanted electrical pulse generator, and a power source such as a battery that may be rechargeable by transcutaneous inductive transfer. An electrode array is connected to the pulse generator, and is positioned adjacent the target neural pathway(s). An electrical pulse applied to the neural pathway by an electrode causes the depolarisation of neurons, and generation of propagating action potentials. The fibres being stimulated in this way inhibit the transmission of pain from that segment in the spinal cord to the brain. To sustain the pain relief effects, stimuli are applied substantially continuously, for example at a frequency in the range of 30 Hz-100 Hz.

For effective and comfortable operation, it is necessary to maintain stimuli amplitude or delivered charge above a recruitment threshold. Stimuli below the recruitment threshold will fail to recruit any action potentials. It is also necessary to apply stimuli which are below a comfort threshold, above which uncomfortable or painful percepts arise due to increasing recruitment of Aβ fibres which when recruitment is too large produce uncomfortable sensations and at high stimulation levels may even recruit sensory nerve fibres associated with acute pain, cold and pressure sensation. In almost all neuromodulation applications, a single class of fibre response is desired, but the stimulus waveforms employed can recruit action potentials on other classes of fibres which cause unwanted side effects. The task of maintaining appropriate neural recruitment is made more difficult by electrode migration and/or postural changes of the implant recipient, either of which can significantly alter the neural recruitment arising from a given stimulus, depending on whether the stimulus is applied before or after the change in electrode position or user posture. There is room in the epidural space for the electrode array to move, and such array movement alters the electrode-to-fibre distance and thus the recruitment efficacy of a given stimulus. Moreover the spinal cord itself can move within the cerebrospinal fluid (CSF) with respect to the dura. During postural changes the amount of CSF and the distance between the spinal cord and the electrode can change significantly. This effect is so large that postural changes alone can cause a previously comfortable and effective stimulus regime to become either ineffectual or painful.

Another control problem, facing neuromodulation systems of all types, is achieving neural recruitment at a sufficient level required for therapeutic effect, but at minimal expenditure of energy. The power consumption of the stimulation paradigm has a direct effect on battery requirements which in turn affects the device's physical size and lifetime. For rechargeable systems, increased power consumption results in more frequent charging and, given that batteries only permit a limited number of charging cycles, ultimately this reduces the implanted lifetime of the device.

Attempts have been made to address such problems by way of feedback, such as by way of the methods set forth in International Patent Publication No. WO2012155188 by the present applicant. Feedback seeks to compensate for nerve and/or electrode movement by controlling the delivered stimuli so as to maintain a constant ECAP amplitude.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” a list of options is to be understood that the element may be any one of the listed options, or may be any combination of two or more of the listed options.

### SUMMARY OF THE INVENTION

According to a first aspect the present invention provides an automated method of controlling a neural stimulus, the method comprising:
applying the neural stimulus to a neural pathway in order to give rise to an evoked action potential on the neural pathway, the stimulus being defined by at least one stimulus parameter;
measuring a neural compound action potential response evoked by the stimulus, and deriving from the measured evoked response a feedback variable;
completing a feedback loop by using the feedback variable to control the at least one stimulus parameter value; and
adaptively compensating for changes in a gain of the feedback loop caused by electrode movement relative to the neural pathway, by applying a compensating transfer function to the feedback variable, the compensating transfer function being configured to compensate for both (i) a distance-dependent transfer function of stimulation, and (ii) a distance dependent transfer function of measurement which is distinct from (i).

According to a second aspect the present invention provides an implantable device for controllably applying a neural stimulus, the device comprising:
a plurality of electrodes including one or more nominal stimulus electrodes and one or more nominal sense electrodes;
a stimulus source for providing a stimulus to be delivered from the one or more stimulus electrodes to a neural pathway in order to give rise to an evoked action potential on the neural pathway;
measurement circuitry for recording a neural compound action potential signal sensed at the one or more sense electrodes; and
a control unit configured to:
   control application of a neural stimulus as defined by at least one stimulus parameter;
   measure via the measurement circuitry a neural compound action potential response evoked by the stimulus;
   determine from the measured evoked response a feedback variable;
   complete a feedback loop by using the feedback variable to control the at least one stimulus parameter value; and
   adaptively compensate for changes in a gain of the feedback loop caused by electrode movement relative to the neural pathway, by applying a compensating transfer function to the feedback variable, the compensating transfer function being configured to compensate for both (i) a distance-dependent transfer function of stimulation and (ii) a distance dependent transfer function of measurement which is distinct from (i).

According to a third aspect the present invention provides a non-transitory computer readable medium for controllably applying a neural stimulus, comprising the following instructions for execution by one or more processors:
computer program code means for applying the neural stimulus to a neural pathway in order to give rise to an evoked action potential on the neural pathway, the stimulus being applied as defined by at least one stimulus parameter;
computer program code means for measuring a neural compound action potential response evoked by the stimulus and deriving from the measured evoked response a feedback variable;
computer program code means for completing a feedback loop by using the feedback variable to control the at least one stimulus parameter value; and
computer program code means for adaptively compensating for changes in a gain of the feedback loop caused by electrode movement relative to the neural pathway, by applying a compensating transfer function to the feedback variable, the compensating transfer function being configured to compensate for both (i) a distance-dependent transfer function of stimulation and (ii) a distance dependent transfer function of measurement which is distinct from (i).

The present invention recognises that (i) recruitment of evoked compound action potentials upon the neural pathway by a given stimulus will vary based on the distance of the stimulus electrode(s) from the neural pathway, and (ii) the observed waveform of a given ECAP from the neural pathway will vary based on the distance of the sense electrode(s) from the neural pathway, and moreover that the transfer function for (i) differs from the transfer function for (ii), so that electrode movement as may be caused by patient movement, postural changes, heartbeat or the like will affect the recruitment behaviour of a system using feedback control of the stimulus.

Some embodiments may provide a control method for effecting constant recruitment by use of single I & V measurements (where I is a measure of stimulus current, and V is a measure of observed ECAP voltage), rather than having to estimate or measure a stimulus threshold or response growth curve. In such embodiments that transfer function preferably comprises a single parameter k reflecting both a recruitment parameter n and a measurement parameter m, m and n being unequal because the stimulation transfer function differs from the measurement transfer function. For example, where the total number of fibres recruited N varies with distance x as ∝Ix⁻ⁿ−T₀, and the measured ECAP voltage amplitude V is approximated as V∝Nx^{-m}, then in some embodiments k=m/n. Importantly, the present invention recognises that the feedback parameter k is not equal to 1, because the stimulation transfer function differs from the measurement transfer function. The feedback parameter k is preferably selected to take a value which is suitable for the stimulation and recording configurations in use, such as a value which depends on a distance from a stimulation electrode(s) to recording electrode(s), and/or which depends on a stimulation electrode to reference electrode configuration, and/or which depends on a stimulation configuration whether bipolar stimulation or tripolar stimulation or the like. For example in one such configuration in which tripolar stimulation is delivered using the first to third electrodes of a SCS lead, and recordings are taken using the sixth electrode of the same lead, the feedback parameter k is preferably selected to be in the range 0-0.8, more preferably 0.1 to 0.7, more preferably 0.16 to 0.61, more preferably 0.22 to 0.53, more preferably 0.3 to 0.43, most preferably about 0.37.

In some embodiments of the invention k is determined clinically using a recruitment datum. The recruitment datum may comprise one or more of the patient's perceptual threshold, discomfort threshold, coverage of a certain area or body part, a qualitative characteristic of the patient's perception of a stimulation such as optimal comfort, an electrophysiological measure, such as the onset of muscle response/twitching, or a measure of neural activity. Such measures may use the amplitude, latency or other characteristic(s) of responses evoked by the stimulus, which may appear in the spine, the peripheral nerves, the brain, or elsewhere in the body. In such embodiments clinical determination of k may comprise the patient assuming a series of postures; in each posture adjusting the stimulus intensity until the required recruitment datum is achieved; and estimating k from constant recruitment data in differing postures by a suitable fitting or approximation.

In some embodiments of the invention k is determined clinically by using the recording electrode to measure neural responses to peripheral stimulation, such as constant TENS stimulation, in a number of postures to obtain Vi data in each posture. The peripheral stimulation may then be removed and the stimulus electrodes of the implant may then deliver stimulation in each posture adjusted to a current level Ii which yields the respective Vi and is thus known to have achieved constant recruitment independently of posture. The set of (Iᵢ, Vᵢ) pairs of constant recruitment may then be fitted or approximated to yield k.

In some embodiments of the invention, particularly for applications involving fibre populations having a narrow range of fibre diameters, k may be determined clinically by placing the patient in a range of postures i, and in each posture sweeping the stimulus intensity and recording a growth curve. From the growth curve for each respective posture, a line is fitted to a linear portion of the curve to determine the threshold Tᵢ and growth slope Mᵢ. Plotting the values of log Tᵢ against log Tᵢ Mᵢ; and fitting a line to these points gives a slope −m/n=−k.

In some embodiments, n and m are treated as constants which approximate the power law applicable to the respective transfer function throughout the operating domain. However, alternative embodiments may provide for variable n and/or variable m, for example to reflect that n and m may reduce slightly, and by differing degrees, with higher recruitment. Such adaptivity in n and m may be implemented so as to provide more accurate compensation depending on whether the device is operating in a regime of low or high recruitment.

Some embodiments of the invention may thus implement a feedback loop as follows: Incoming measurements of ECAPs (V) are multiplied with an exponentiated version (I^{-k}) of the stimulus current (I) used to generate them. An error signal is generated relative to a setpoint and fed into a controller G, which determines the next stimulus intensity. I-V control can thus be implemented as a feedback loop, controlling a term of the form F=I^{k}V with error signal e=\hat{F}-F derived from the chosen setpoint \hat{F}.

The feedback variable could in some embodiments be any one of: an amplitude; an energy; a power; an integral; a signal strength; or a derivative, of any one of: the whole evoked compound action potential; the fast neural response for example in the measurement window 0-2 ms after stimulus; the slow neural response for example in the measurement window 2-6 ms after stimulus; or of a filtered version of the response. The feedback variable could in some embodiments be an average of any such variable determined over multiple stimulus/measurement cycles. The feedback variable may in some embodiments be the zero intercept, or the slope, of a linear portion of the response of ECAP amplitude to varying stimulus current. In some embodiments the feedback variable may be derived from more than one of the preceding measures.

The control variable, or stimulus parameter, could in some embodiments be one or more of the total stimulus charge, stimulus current, pulse amplitude, phase duration, interphase gap duration or pulse shape, or a combination of these.

The present invention thus recognises that using a feedback loop to maintain a constant ECAP is a difficult task as changes in patient posture both create signal inputs and change the loop characteristics of both (i) the stimulation electrode to nerve transfer function, and (ii) the nerve to sense electrode transfer function.

The set point of the feedback loop may be configured so as to seek a constant value of ECAP amplitude, or may be configured to seek a target ECAP amplitude which changes over time, for example as defined by a therapy map as described in International Patent Application Publication No. WO2012155188 by the present applicant, the content of which is incorporated herein by reference.

### BRIEF DESCRIPTION OF THE DRAWINGS

An example of the invention will now be described with reference to the accompanying drawings, in which:

FIG. 1 schematically illustrates an implanted spinal cord stimulator;

FIG. 2 is a block diagram of the implanted neurostimulator;

FIG. 3 is a schematic illustrating interaction of the implanted stimulator with a nerve;

FIG. 4 illustrates the typical form of an electrically evoked compound action potential;

FIG. 5 is a plot of the peak-to-peak amplitude of observed ECAPs;

FIG. 6 is a plot of recruitment as a function of current;

FIG. 7 illustrates recruitment as a function of recording amplitude;

FIG. 8 illustrates the corresponding stimulus current for each distance x;

FIG. 9 shows voltage-distance curves calculated from the ECAP model;

FIG. 10 illustrates the relationship between I^{k}V value and recruitment;

FIGS. 11a and 11b illustrate performance of the previous constant current approach, the previous constant ECAP voltage approach, and the I-V approach;

FIG. 12 illustrates performance of the I-V approach across a range of desired recruitment values;

FIG. 13 illustrates the overall error for each value of k;

FIG. 14 illustrates variation in k between electrodes;

FIG. 15 shows the effect of different values of k on the performance of the I-V technique, with biphasic stimulation and on different electrodes;

FIG. 16 shows the performance of I, V, and I-V control with biphasic stimulation; and

FIG. 17 illustrates a feedback loop to maintain constant recruitment, using I-V control.

### DESCRIPTION OF THE PREFERRED EMBODIMENTS

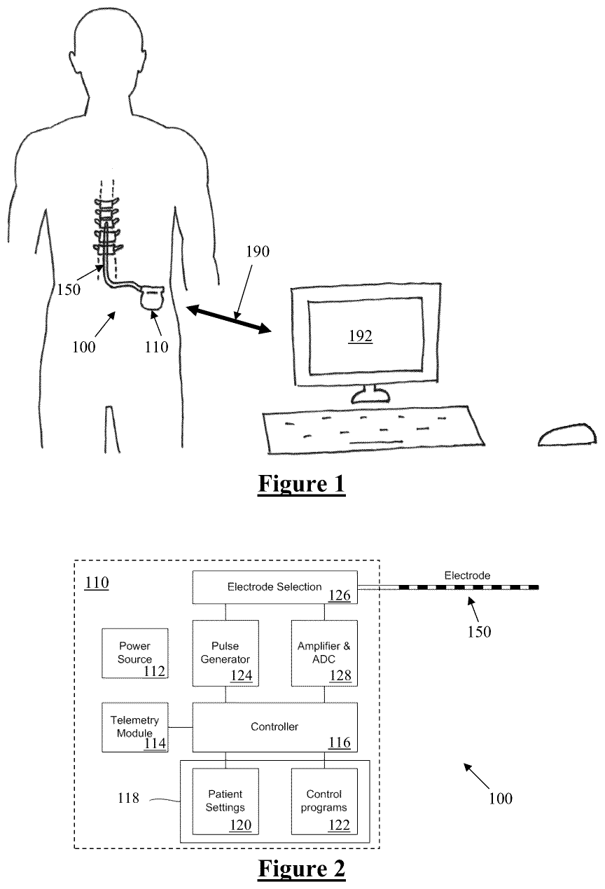

FIG. 1 schematically illustrates an implanted spinal cord stimulator 100. Stimulator 100 comprises an electronics module 110 implanted at a suitable location in the patient's lower abdominal area or posterior superior gluteal region, and an electrode assembly 150 implanted within the epidural space and connected to the module 110 by a suitable lead. Numerous aspects of operation of implanted neural device 100 are reconfigurable by an external control device 192. Moreover, implanted neural device 100 serves a data gathering role, with gathered data being communicated to external device 192.

FIG. 2 is a block diagram of the implanted neurostimulator 100. Module 110 contains a battery 112 and a telemetry module 114. In embodiments of the present invention, any suitable type of transcutaneous communication 190, such as infrared (IR), electromagnetic, capacitive and inductive transfer, may be used by telemetry module 114 to transfer power and/or data between an external device 192 and the electronics module 110.

Module controller 116 has an associated memory 118 storing patient settings 120, control programs 122 and the like. Controller 116 controls a pulse generator 124 to generate stimuli in the form of current pulses in accordance with the patient settings 120 and control programs 122. Electrode selection module 126 switches the generated pulses to the appropriate electrode(s) of electrode array 150, for delivery of the current pulse to the tissue surrounding the selected electrode(s). Measurement circuitry 128 is configured to capture measurements of neural responses sensed at sense electrode(s) of the electrode array as selected by electrode selection module 126.

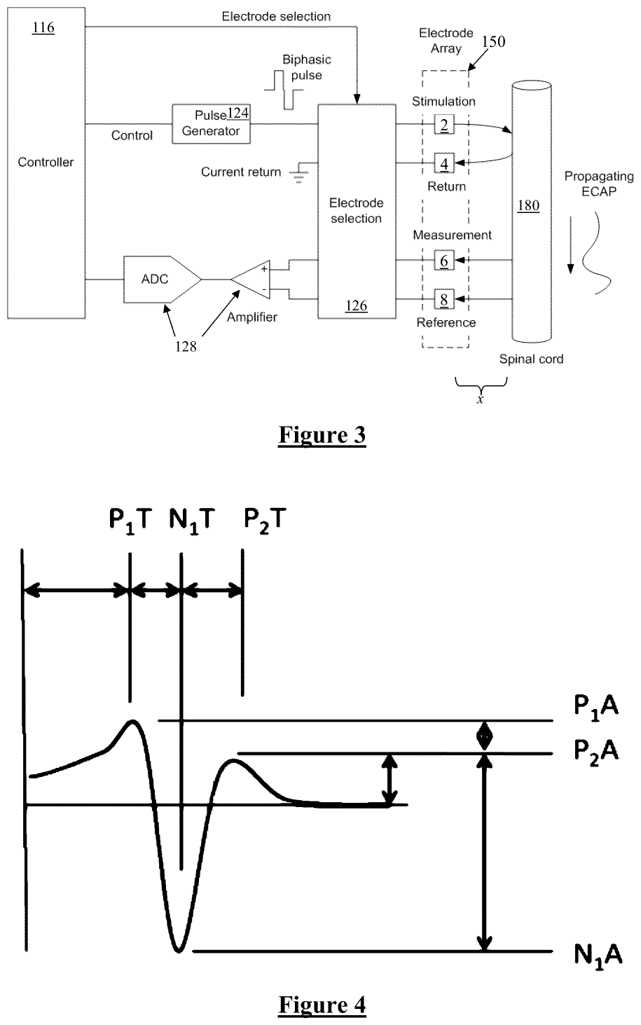

FIG. 3 is a schematic illustrating interaction of the implanted stimulator 100 with a nerve 180, in this case the spinal cord however alternative embodiments may be positioned adjacent any desired neural tissue including a peripheral nerve, visceral nerve, parasympathetic nerve or a brain structure. Electrode selection module 126 selects a stimulation electrode 2 of electrode array 150 to deliver an electrical current pulse to surrounding tissue including nerve 180, and also selects a return electrode 4 of the array 150 for stimulus current recovery to maintain a zero net charge transfer.

Delivery of an appropriate stimulus to the nerve 180 evokes a neural response comprising a compound action potential which will propagate along the nerve 180 as illustrated, for therapeutic purposes which in the case of a spinal cord stimulator for chronic pain might be to create paraesthesia at a desired location. To this end the stimulus electrodes are used to deliver stimuli at 30 Hz. To fit the device, a clinician applies stimuli which produce a sensation that is experienced by the user as a paraesthesia. When the paraesthesia is in a location and of a size which is congruent with the area of the user's body affected by pain, the clinician nominates that configuration for ongoing use.

The device 100 is further configured to sense the existence and intensity of compound action potentials (CAPs) propagating along nerve 180, whether such CAPs are evoked by the stimulus from electrodes 2 and 4, or otherwise evoked. To this end, any electrodes of the array 150 may be selected by the electrode selection module 126 to serve as measurement electrode 6 and measurement reference electrode 8. Signals sensed by the measurement electrodes 6 and 8 are passed to measurement circuitry 128, which for example may operate in accordance with the teachings of International Patent Application Publication No. WO2012155183 by the present applicant, the content of which is incorporated herein by reference.

The present invention recognises that in attempting to implement a feedback control loop, there are two distance-dependent transfer functions involved in ECAP recording. The first is in stimulation: at a greater distance x, a higher current is needed to stimulate the same nerve fibres. The second is in recording: at a greater distance x, a given neural recruitment results in a smaller observed ECAP. Feedback seeking a constant observed ECAP voltage amplitude takes no account of the recording transfer function, with the result that recruitment will actually increase as the cord distance increases. Moreover, the first and second transfer functions are unequal and require separate compensation.

The present invention provides an approach which considers both distance-dependent transfer functions, in a manner which is responsive to the differences between the transfer functions, to thereby improve the performance of feedback control.

Such a method is necessarily limited by the impossibility or at least impracticality of directly measuring neural recruitment in humans via intracellular patch clamp recording or the like; it must be possible in a practical feedback system to fit feedback parameters to the patient without such measurements.

FIG. 4 illustrates the typical form of an electrically evoked compound action potential of a healthy subject. The shape of the compound action potential shown in FIG. 4 is predictable because it is a result of the ion currents produced by the ensemble of axons generating action potentials in response to stimulation. The action potentials generated among a large number of fibres sum to form a compound action potential (CAP). The CAP is the sum of responses from a large number of single fibre action potentials. The CAP recorded is the result of a large number of different fibres depolarising. The propagation velocity is determined largely by the fibre diameter. The CAP generated from the firing of a group of similar fibres is measured as a positive peak potential P₁, then a negative peak N₁, followed by a second positive peak P₂. This is caused by the region of activation passing the recording electrode as the action potentials propagate along the individual fibres. An observed CAP signal will typically have a maximum amplitude in the range of microvolts.

The CAP profile takes a typical form and can be characterised by any suitable parameter(s) of which some are indicated in FIG. 4. Depending on the polarity of recording, a normal recorded profile may take an inverse form to that shown in FIG. 4, i.e. having two negative peaks N₁ and N₂, and one positive peak P₁.

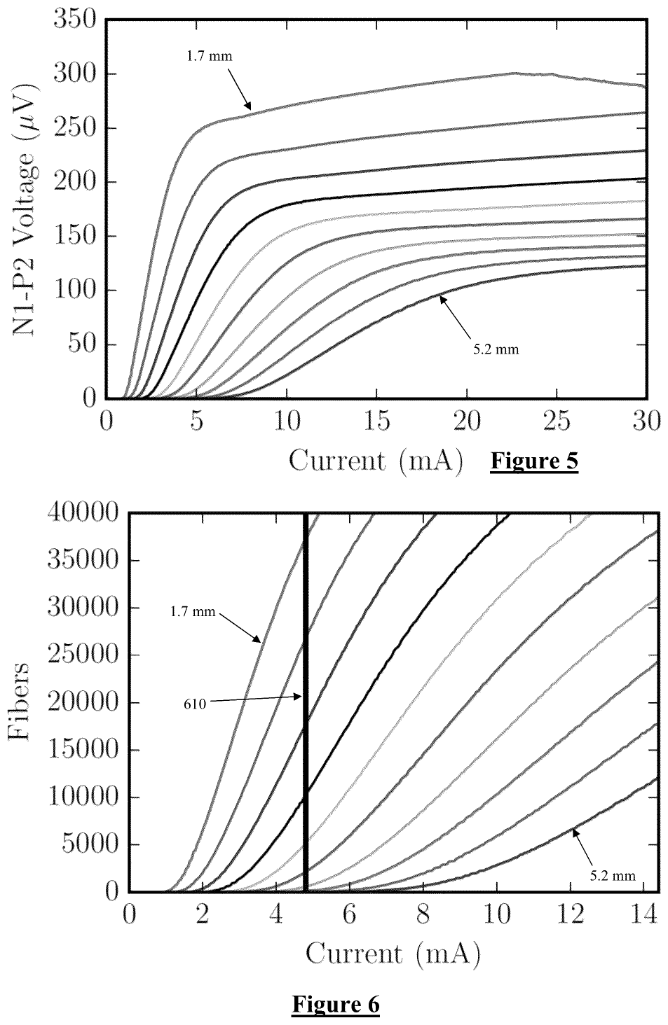

For purposes of illustration, ECAP simulations were performed with an SCS model for each of 10 different cord positions, varying the cord-electrode distance from 1.7 mm to 5.2 mm (1.7 mm, 2.1 mm, 2.5 mm, 2.9 mm, 3.3 mm, 3.6 mm, 4.0 mm, 4.4 mm, 4.8 mm, 5.2 mm). Monophasic stimuli were used to avoid confounding measurements with the second-cathode effect. An 8-electrode linear array is modelled, as is commonly used in SCS. Stimuli are delivered on electrode 2 with current returned on electrodes 1 and 3. FIG. 5 is a plot of the peak-to-peak amplitude of the ECAPs observed on electrode 6 at these cord-electrode distances in response to stimulus currents in the range of 0-30 mA. As can be seen, as the cord is moved closer to the electrode array, the recruitment threshold is lowered and the growth slope and saturation amplitude increase.

In order to compare stimulation methods, a target recruitment of 5000 fibres is chosen; this is within the linear recruitment region, as commonly observed in therapeutic stimulation. FIG. 6 is a plot of recruitment as a function of current, illustrating the use of a fixed stimulus amplitude (vertical line 610) in accordance with prior art approaches. The fixed current amplitude (approx. 4.8 mA) is chosen to recruit 5000 fibres at the medial cord position. The number of fibres recruited using a constant stimulus current ranges from zero fibres to nearly 35000 fibres across the examined cord positions. This highlights the degree to which traditional SCS is sensitive to cord position.

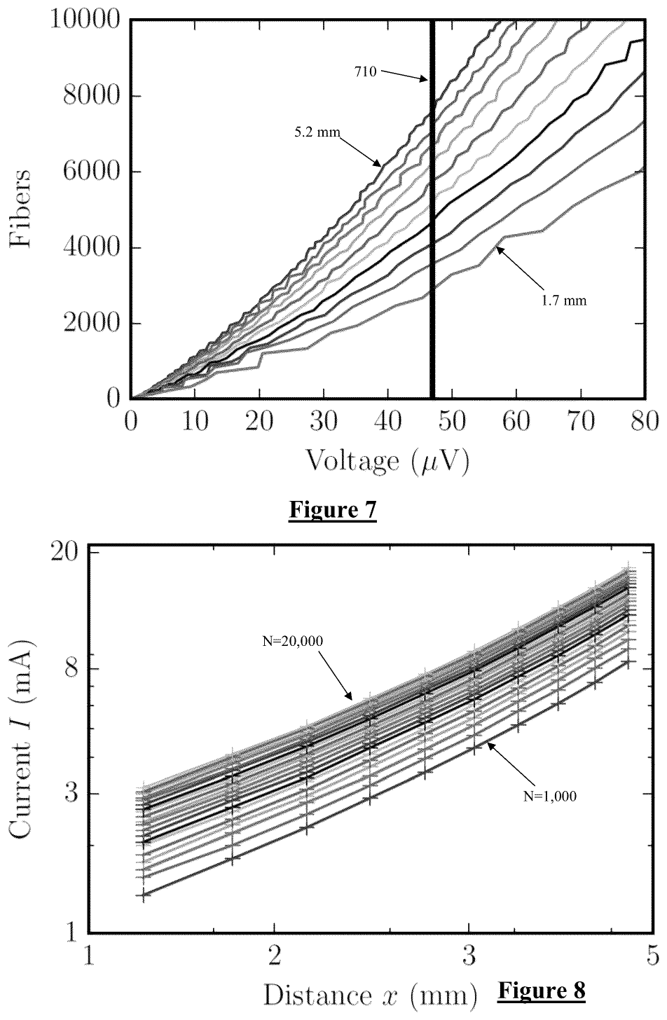

FIG. 7 illustrates recruitment as a function of recording amplitude, when using feedback control which seeks a constant N1-P2 recording amplitude (vertical line 710). The set amplitude (approx. 47 μV) is chosen so as to recruit 5000 fibres at the medial cord position. Compared to the constant stimulus amplitude approach of FIG. 6, the recruitment variation resulting from electrode-nerve separation changes in FIG. 7 is reduced, with recruitment now varying from approximately 3000 to 6500 fibres across the distances examined. It is noted that the variation in FIG. 7 is reversed as compared to the constant-current stimulation approach of FIG. 6: in FIG. 7 the recruitment is increased at larger cord distances. Nevertheless, the constant observed voltage approach of FIG. 7 continues to suffer from a considerable degree of undesired recruitment variation in response to changes in electrode-to-nerve separation, with recruitment being almost 40% less than desired when electrode-to-nerve separation is 1.7 mm, and being almost 60% greater than desired when electrode-to-nerve separation is 5.2 mm.

The present invention thus recognises that when the electrode-to-nerve separation is subject to change, the constant stimulus approach of FIG. 6 and the constant observed voltage approach of FIG. 7 both fail to reliably recruit the desired therapeutic number of neural fibres.

The present invention instead provides for feedback control of stimulus amplitude in a manner which compensates for both (i) the stimulus transfer function relative to electrode-to-nerve separation x and (ii) the recording transfer function relative to electrode-to-nerve separation x, and does so in a manner which accounts for the differences between (i) and (ii)

Both the stimulus transfer function and the recording transfer function describe a physical process where a first element (stimulation electrode or nerve, respectively) radiates an electric field in a volume conductor, and some of this field is sensed by a second element (nerve or sense electrode, respectively). The coupling of a radiative process typically falls off with some power of the distance, and can be modelled by equations including such a power term. Importantly, however, the stimulation transfer function is not the same as (nor the inverse of) the measurement transfer function, at least due to the differing originating waveforms (pulsatile stimulus vs. a typically 3-lobed ECAP waveform), due to the differing electrode configurations invariably employed to deliver stimulation on the one hand and to obtain neural measurements on the other hand, and due to the increasing dispersion of the ECAP waveform as it travels away from the stimulation site, at least.

In order to address the unequal transfer functions appropriately, we first derive an expression for the relationship between the stimulus intensity/and the number of recruited fibres N, i.e. the stimulation transfer function. This function will depend on the distance x between the target tissue and the stimulating electrodes. N will be equal to the number of fibres with thresholds lower than the stimulus intensity; these thresholds Tᵢ will also vary with distance:

$$\\ N = {\sum }_{i} [ I > {T}_{i} ( x ) ]$$

The change in Tᵢ with x can be approximated by a simple analytic model. If we consider a single myelinated nerve fibre exposed to a point current source at distance x and with internodal length L, the voltage at the qth node of Ranvier is of the form:

$$\\ \frac{I}{\sqrt{{x}^{{}_{} 2} + {( qL )}^{2}}}$$

where q∈ [symbol] and assuming that the 0^{th} node is at the point on the fibre nearest the electrode.

The propensity of the fibre to be activated by a given stimulus is approximated by a function known as the activating function. This represents the net depolarisation current being applied to each node of Ranvier on the fibre, and has a threshold behaviour; if the depolarisation is sufficient at any node, the fibre will fire. For a myelinated fibre, the activating function is given by the second difference of the field along the fibre. This has a maximum at the node nearest the electrode, with value

$$\\ I {}_{} ( \frac{2}{\sqrt{{x}^{{}_{} 2}}} - \frac{1}{\sqrt{{x}^{{}_{} 2} + {L}^{2}}} - \frac{1}{\sqrt{{x}^{{}_{} 2} - {L}^{2}}} )$$

Thus the threshold will vary with distance as

$$\\ {T}_{i} ∝ {( \frac{1}{x} - \frac{1}{\sqrt{{x}^{{}_{} 2} + {L}^{2}}} )}^{- 1}$$

This is not a particularly tractable expression. The internodal spacing in the dorsal columns is generally less than the cord-electrode distance; in the region L<x, the fourth and higher derivatives of Tᵢ are quite small, and the behaviour approximates

Tᵢ∝xⁿ

For the case of the monopolar point source stimulation, n∈[1,3]. In other configurations of electrodes, surrounding tissue, and nerves, the value of n may be outside this range.

Applying this to the ensemble behaviour, the total number of fibres recruited by a given stimulus depends on the fibre thresholds Tᵢ. In the linear region of recruitment growth, N increases linearly with I, so the Tᵢ can be assumed uniformly distributed, and the number recruited varies as

$$\\ N ∝ {Ix}^{{}_{} - n} - {T}_{0}$$

where T₀ is a normalised threshold corresponding to the threshold of the most sensitive fibres at x=1.

From this derivation, it can be seen that the power n (also referred to as the stimulation transfer function parameter) will depend on the electrical and geometric relationship between stimulation electrodes and stimulated tissue. For example, with the use of multipolar stimulus electrode configurations as commonly found in therapeutic SCS, the near-field activating function may be increased due to the increased variation in the field, while the far-field may fall off more quickly due to dipole cancellation. These effects also depend on the geometry of the stimulated nerve fibres, and on the electrical properties of the intervening and surrounding tissues; for example, the longitudinal anisotropic conductivity of the white matter of the dorsal columns affects the field shape. The stimulus intensity/in the presently described embodiments is stimulus current, although alternative stimulus intensity parameters (voltage, pulse width, etc) may equivalently be used.

This relationship is examined using the ECAP model results. For a given recruitment value N, the corresponding stimulus current for each distance x is calculated. This is shown in FIG. 8 for recruitment values up to N=20,000, within the linear portion of the recruitment curve. The lines in FIG. 8 are not exactly straight, indicating that recruiting the same number of fibres at larger distances requires disproportionately higher current, indicating a shift in the power law. Treating n as a constant allows the slopes of the curves to be used as an estimate for n: using linear regression over log-log axes results in n≈1.64 at low recruitment (N=1,000), dropping to n≈1.55 at higher recruitment levels (N=20,000).

As noted in the preceding, it is necessary to not only address the stimulation transfer function, it is also necessary to address the recording transfer function. Thus, we derive an expression for the recording transfer function; being the relationship between the number of recruited fibres N and the observed N₁-P₂ ECAP amplitude V, although other measures of ECAP intensity may equivalently be used. The recorded signal V varies in a distance-dependent manner with the neural recruitment. The action potential results from a region of depolarisation which effectively propagates between nodes of Ranvier; this also results in membrane currents both ahead of and behind the depolarisation, effectively producing a field commensurate with a double dipole or tripole source. The propagation of an action potential along a myelinated fibre is too complex for meaningful analytical treatment; instead, simulation can be undertaken of a collection of myelinated nerve fibres with a point recording electrode, for which it has been found that the single fibre action potential (SFAP) amplitude Sᵢ of a single fibre at distance x followed a law

$$\\ {S}_{i} ∝ {x}^{{}_{} - m}$$

where m=1 close to the fibre, and m=3 in the far field. The former is expected where x<<L, and the nearest node's action current dominates the recording; the latter results from the approximately tripolar nature of the travelling action potential.

The ECAP voltage V results from the summation of many single fibre action potentials (SFAPs), and thus depends on the spatial and diametric distribution of recruited fibres. Different diameter fibres have different SFAP amplitudes, however the present embodiment of the invention notes that their proportions are fairly constant over the linear portion of the growth curve. The present embodiment of the invention further assumes that the spatial distribution of recruited fibres varies less than x, which enables us to approximate the ECAP amplitude as:

$$\\ \begin{matrix}V ∝ {Nx}^{{}_{} - m} & ( 2 )\end{matrix}$$

where m is a recording transfer function parameter.

In practice, the recording electrodes are not points, but physical structures of significant dimensions compared to the nerves and/or nerve-electrode distance. Differential recording is often used, where the ECAP is measured as a difference in potential between two electrodes in proximity to the target tissue. The ECAP also undergoes changes in waveform as it propagates away from the point of initiation due to dispersion, fibre termination and so forth. The surrounding electrical environment also affects the recording transfer. The membrane properties of the nerve fibres also affect the depolarisation behaviour and hence the induced external currents. These and other factors introduce additional influences on m.

From these derivations, it can be seen that n depends on factors including the stimulus electrode configuration, including configuration of drive and return electrodes; dimension and placement of electrodes; conductive properties of surrounding tissues; and the nerve fibre geometries. Meanwhile, m depends on factors including the recording electrode (and reference electrode, if used); the membrane properties and geometries of the individual nerve fibres; the overall neural population stimulated; and the surrounding tissues. Thus it is expected that m and n will take different values, and may further vary with the current cord-electrode distance.

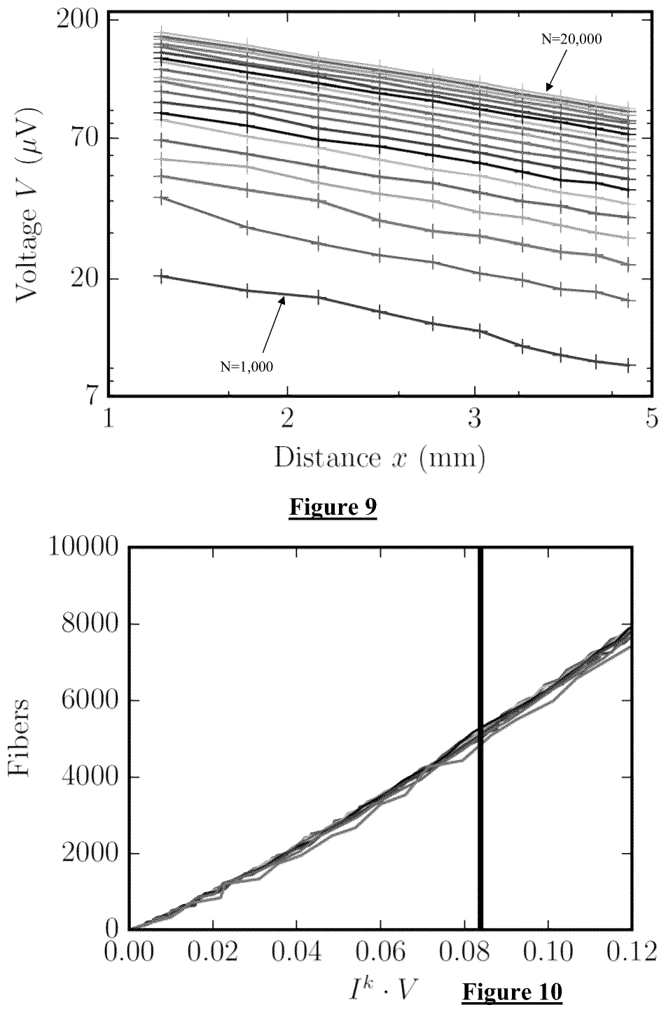

The recording transfer function in the ECAP model is examined using voltage distance curves at constant recruitments. FIG. 9 shows voltage-distance curves calculated from the ECAP model results. Each line shows the relationship between ECAP amplitude and distance, for a fixed recruitment ranging from N=1000 (bottom curve) to N=20,000 (top curve) in steps of 1000, on log-log axes. The slope of each line is an estimate of the value −m. These curves indicate values for m of about 0.75 at the beginning of recruitment (small N), with m dropping to about 0.6-0.65 over the linear region. Without intending to be limited by theory, it may be that the decreasing value of m corresponds to the broadening of the range of recruited fibre diameters, whereby the diameter-dependent conduction velocity of myelinated fibres results in a dispersion of the action potential volley; this results in a longer region of the nerve trunk that is contributing to the ECAP via membrane currents. The triphasic field radiated by each fibre then cancels somewhat, resulting in a field that falls off more rapidly with distance.

The stimulation transfer function parameter n and the recording transfer function parameter m can be determined as they are related to the electrode geometry and configuration and are not expected to change appreciably during therapy. The distance x is not known, however the stimulation transfer function and the recording transfer function can be combined to compensate for the changes in x and ensure constant recruitment. The stimulation current I can be known, and the ECAP voltage V can be measured, on each stimulus. Substituting equation (2) into equation (1) gives:

$$\\ \begin{matrix}N ∝ I {}_{} {( \frac{AN}{V} )}^{- n / m} - {T}_{0} & ( 3 )\end{matrix}$$

where A is the constant of proportionality of equation 2.

In order to maintain constant N, as is desired for feedback control, it follows that I^{m}/ⁿV must be constant. For the purpose of recruitment control, this is most easily expressed as:

$$\\ \begin{matrix}y = {I}^{{}_{} k} V & ( 4 )\end{matrix}$$

where k=m/n and k>0.

This embodiment of the invention thus captures the transfer function behaviour in a single parameter k which, being derived from m and n, reflects both the stimulation transfer function and the recording transfer function and, importantly, reflects that these transfer functions are not the same, as discussed in the preceding, and is configured to compensate for both unique transfer functions.

The stimulus current I can then be adjusted using any suitable feedback algorithm (such as is shown in FIG. 17, discussed further below) to maintain the relationship between I and V, with the desired value of y being driven to a chosen set-point. A higher value of y will result in higher recruitment.

The performance of this feedback method is examined by measuring the setpoint-recruitment curve for various distances. For this comparison, m and n were estimated to be 0.6 and 1.6, respectively, giving a value of k=0.37. The relationship between I^{k}V value and recruitment is shown in FIG. 10.

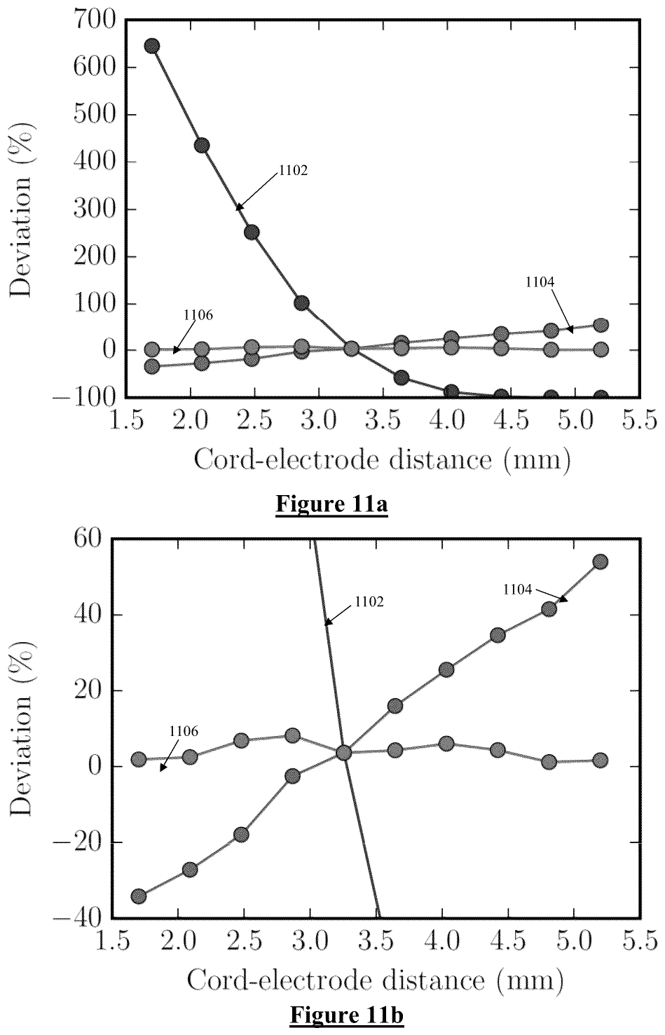

The setpoint shown in the figure is chosen for N=5,000 fibre recruitment at a cord-electrode distance x of 3.2 mm, in the middle of the range. Across the full range of cord positions, ranging from 1.7 mm to 5.2 mm, the recruitment remains within a narrow range demonstrating the benefit of this embodiment of the invention, and illustrating that an assumption of n and m being constant performs well. FIG. 11a illustrates performance of the previous constant current approach 1102, the previous constant ECAP voltage 1104, as compared to the present embodiment 1106. The extremely poor performance of constant-current stimulation 1102 is visible in the full view of FIG. 11a; the detail view of FIG. 11b shows the improved performance of the present embodiment's I-V control 1106 over constant-amplitude control 1104. Constant-amplitude control 1104 results in variations of greater than −30/+50% from the setpoint, while I-V control 1106 maintains constant recruitment to better than ±5%.

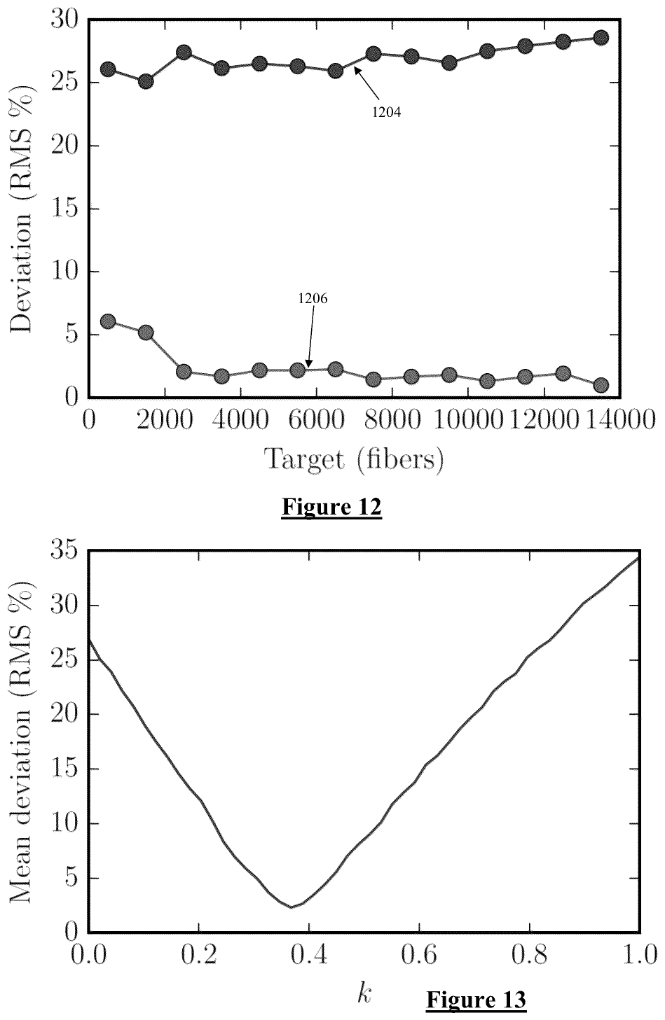

Moreover, the I-V control 1206 performs consistently well across a range of desired recruitment values, as shown in FIG. 12. For each desired recruitment, a suitable setpoint was determined for each algorithm, again using the cord distance of 3.2 mm. The recruitment was calculated for each cord position with that setpoint, and the RMS deviation from the mean recorded. I-V control 1206 results in less variation in recruitment (typically <5%) across a wide range of setpoints as compared to the previous constant ECAP approach 1204 (typically >25%).

The I-V control method of the present embodiment requires that a suitable value for k to be chosen. A sensitivity analysis was conducted, varying k between 0 and 1. For each value, an RMS deviation measurement was made of the type shown in FIG. 12. The overall error for each value of k was estimated by averaging the RMS deviation for all considered recruitment levels, shown in FIG. 13. With k=0, the current term (I) is removed from the feedback equation, making I-V control equivalent to constant-amplitude control with relatively poor mean deviation (RMS) of over 25%. FIG. 13 shows that I-V control outperforms constant-amplitude control for any value of k between 0 and 0.8.

In such a simulation, suitable ranges of k to achieve given performance can be read directly off FIG. 13, for example to achieve mean deviation less than 20% k should be set 0.1 to 0.7, less than 15% mean deviation gives k 0.16 to 0.61, less than 10% mean deviation gives k 0.22 to 0.53, less than 5% mean deviation gives k 0.3 to 0.43, least mean deviation gives k about 0.37. However, in practice the plot of FIG. 13 is not available and indirect measures are required in order to determine k in order to fit an implant to a recipient, as discussed further below.

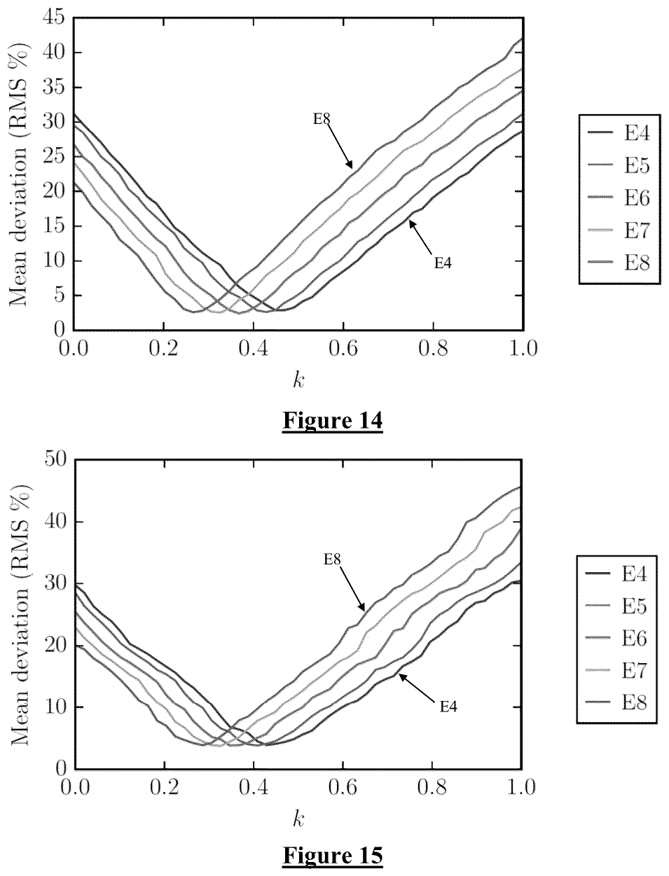

The recording transfer function of equation (2) depends on the recording electrode in use. Geometric factors may differ between electrodes, and the dispersion of the action potential volley increases as it travels away from the stimulation site. Increasing dispersion decreases m, so the correct value of k can be expected to be lower with increasing distance from the stimulus. This is seen in FIG. 14. If recording on electrode 4, closest to the stimulus, the optimal k is approximately 0.45, but any value between 0 and 1 outperforms constant-amplitude feedback. For electrode 8, the optimal value is approximately 0.27, with values between 0 and 0.55 outperforming constant-amplitude.

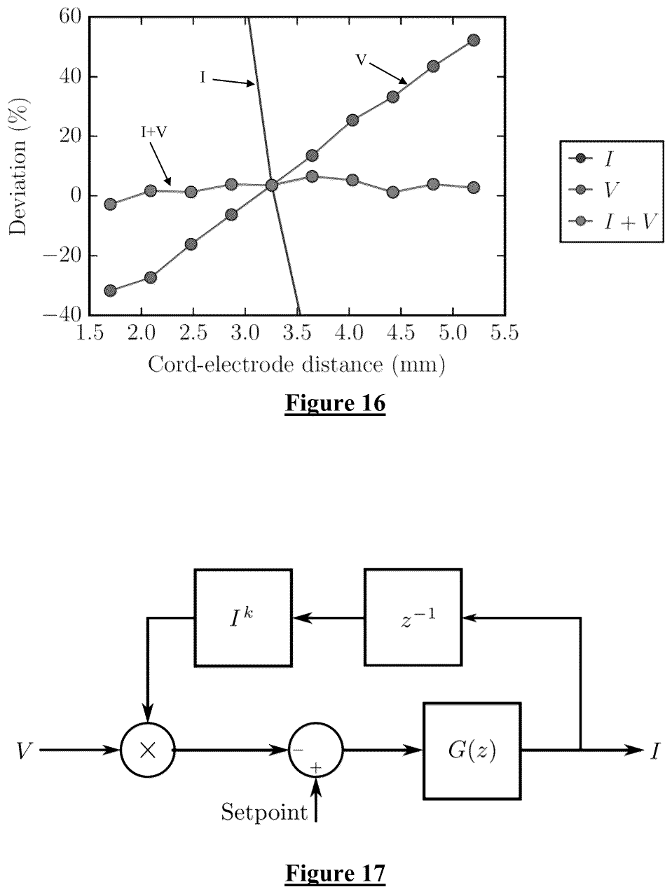

The results of FIGS. 5 to 14 were prepared using monophasic stimulation, in order to clearly demonstrate the principles without consideration of the second cathode effect and its complication of results. However, the techniques described are equally applicable when biphasic stimulation is used, as shown in FIGS. 15 and 16. In particular, FIG. 15 shows the effect of different values of k on the performance of the present technique of recruitment control, with biphasic stimulation and on different electrodes. The best results obtained in the biphasic stimulus case are similar to those in the monophasic case, although the optimal value for k is somewhat lower. I-V control in the biphasic case outperforms constant-amplitude control for k values between 0 and 0.65. FIG. 16 shows the performance of I, V, and I-V control with biphasic stimulation. A k-value of 0.35 was used for I-V control. Voltage mode (V) control results in a variation of greater than −30/+50% across the examined range of distances, while I-V control maintains recruitment within less than +10/−0% of the initial setpoint.

The I-V control method thus requires a single parameter, k, which will depend on the patient's spinal geometry as well as the stimulation and recording configuration. In some embodiments of the invention, k can be determined from a precomputed table or almanac, where a fixed value is chosen based on one or more of the stimulation and recording parameters. For example, the distance between stimulation and recording electrode, the stimulus pulse-width, and the location of the measurement reference electrode may be used to determine the optimal value of k.

In some embodiments of the invention k can be determined clinically, using a recruitment datum. A recruitment datum can be used as a reference point to adjust the stimulus intensity and achieve the same level of neural recruitment in different postures. Suitable data may include the patient's perceptual threshold, discomfort threshold, coverage of a certain area or body part, or any qualitative characteristic of the patient's perception of a stimulation such as optimal comfort. Electrophysiological measures may also be used, such as the onset of muscle response/twitching, or some measure of neural activity. Such measures may use the amplitude, latency or other characteristic(s) of responses evoked by the stimulus, which may appear in the spine, the peripheral nerves, the brain, or elsewhere in the body.

In this embodiment, the patient is instructed to assume a series of postures, achieving a different but unknown cord-electrode distance xᵢ in each. In each posture, the stimulus intensity is adjusted until the recruitment datum is achieved. The resulting current Iᵢ and ECAP measure Vᵢ are recorded for each posture i. Since the use of the recruitment datum implies that N is constant across these measurements, this implies that

$$\\ {I}_{i} ∝ {x}_{i}^{{}_{} n}$$

$$\\ {V}_{i} ∝ {x}_{i}^{{}_{} - m}$$

Thus, one simple method to estimate k is to plot log Iᵢ against log Vᵢ and fit a line to these data points; this line would then have a slope of −n/m=−k. Other methods for approximating solutions to such equations may also be used.

Other methods which recruit a constant subpopulation of nerve fibres may also be used for this task. For example, peripheral nerve stimulation using transdermal electrical nerve stimulation (TENS) can provide constant recruitment of a peripheral nerve, even as the patient's posture changes; if some subset of these peripheral nerves extend into the spine in the vicinity of the recording electrode, then an evoked signal Vᵢ can be recorded in each posture i. After recording Vᵢ in each posture, the peripheral stimulus is removed, and the therapeutic stimulus introduced; its intensity is adjusted to reproduce an evoked response of amplitude equal to Vᵢ, and this intensity Iᵢ recorded. This procedure produces a set of (Iᵢ, Vᵢ) pairs of constant recruitment, which may be fitted for k as with other recruitment datum data.

Alternative embodiments may however seek to determine k directly from ECAP recordings, without reference to the patient's percept. The recording transfer function between recruitment, distance, and ECAP amplitude is complex, particularly as it depends on the dispersion characteristics of the recruited fibre population. Amplitude measurements alone cannot always distinguish changes in dispersion from changes in recruitment. However, a fitting technique is presented here which is suitable for fibre populations which have a very narrow range of diameters. Although this is not the case in the spinal column the following technique may be useful elsewhere in the body when using a single recording electrode. In some cases, particularly where the fibre population is fairly homogeneous, it is possible to determine k from threshold and slope measurements of the growth curve.

In a typical growth curve, there is a linear region where the measured variable grows linearly with the stimulus intensity. Therapeutic SCS operates in the linear region. The growth curves shown in FIG. 5 show such a linear region; for example at a cord-electrode distance of 1.7 mm, this region extends between 1.5 mA and 3 mA; at 5.2 mm, this extends approximately between 9 mA and 18 mA. Such a linear region can be characterised by fitting a line to it; it is then described by the slope M and the x-intercept (threshold) T of that line. The ECAP measurement V in this linear region can then be modelled as a function of stimulus intensity I:

$$\\ V = M ( I - T )$$

It is then apparent, when considering the power law models of stimulation and recording transfer functions, that

$$\\ T ∝ {x}^{{}_{} n}$$

$$\\ M ∝ {x}^{{}_{} - ( m + n )}$$

Thus, a method for estimating k is to place the patient in a range of postures i, and in each posture, sweep the stimulus intensity and record a growth curve. From each growth curve, a line is fitted determining the threshold Tᵢ and growth slope Mᵢ. The values of log Tᵢ can be plotted against log Tᵢ Mᵢ; a line fitted to these points then has slope −m/n=−k. Other methods for finding solutions to these equations may alternatively be used.

FIG. 17 illustrates a feedback loop to maintain constant recruitment, using I-V control in accordance with the present embodiment. Incoming measurements of ECAPs (V) are multiplied with an exponentiated version (I^{k}) of the stimulus current (I) used to generate them. An error signal is generated relative to the setpoint and fed into a controller G, which determines the next stimulus intensity. I-V control can thus be implemented as a feedback loop in the manner shown. The setpoint is a unitless value determined by the patient's desired level of stimulation. A discrete-time controller G(z) is used to control the stimulus current based on the error signal; this may take the form of a simple gain or a more complex system such as a PID controller.

This particular scheme has the potential to improve loop response speed in addition to providing better control accuracy. In constant-amplitude feedback, the patient's transfer function (from stimulus to ECAP amplitude) varies with posture; this limits the maximum controller gain that can be applied while keeping the loop stable, and in doing so limits the potential bandwidth. If I-V control is implemented by scaling the ECAP amplitude at the input to G(z), then when the loop is tracking correctly the scaling compensates for the changing transfer function of the patient. This means that the gain of G(z) can be maximised without compromising stability, increasing the speed with which the loop can respond.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not limiting or restrictive.

1. An automated method of controlling a neural stimulus, the method comprising:
applying, via one or more stimulus electrodes of a plurality of implanted electrodes, the neural stimulus to a neural pathway of a patient in order to evoke a neural compound action potential response on the neural pathway, the neural stimulus being defined by a value (I) of stimulus intensity;
measuring, via one or more sense electrodes of the plurality of implanted electrodes, a neural compound action potential response evoked by the neural stimulus,
deriving, from the measured evoked response and the value (I) of the stimulus intensity, a feedback variable that is a proxy for a level of recruitment of neurons on the neural pathway by the neural stimulus, independent of the distance between the implanted electrodes and the neural pathway; and
completing a feedback loop by using the feedback variable to control the stimulus intensity.

2. The method of claim 1 wherein the deriving uses a value (V) of measured amplitude of the evoked neural compound action potential response.

3. The method of claim 2 wherein the deriving uses a parameter k reflecting both a recruitment parameter n and a measurement parameter m, where m is not equal to n.

4. The method of claim 3 wherein the value of the parameter k is selected based on the stimulation and recording configurations in use.

5. The method of claim 3 wherein the value of the parameter k is determined clinically using a recruitment datum.

6. The method of claim 5 wherein the recruitment datum comprises one or more of: the patient's perceptual threshold, the patient's discomfort threshold, coverage of a certain area or body part, a qualitative characteristic of the patient's perception of a stimulation, the patient's perception of optimal comfort, an electrophysiological measure, the onset of muscle response/twitching, and a measure of neural activity.

7. The method of claim 1 wherein completing the feedback loop comprises:
generating an error signal from the feedback variable relative to a setpoint, and
determining the next stimulus intensity value from the error signal.

8. An implantable device for controllably applying a neural stimulus, the device comprising:
a plurality of electrodes including one or more stimulus electrodes and one or more sense electrodes;
a stimulus source for providing a stimulus to be delivered from the one or more stimulus electrodes to a neural pathway of a patient in order to evoke a neural compound action potential response on the neural pathway;
measurement circuitry for processing a neural compound action potential response signal sensed at the one or more sense electrodes; and
a control unit configured to:
control application of a neural stimulus as defined by a value (I) of stimulus intensity;
measure via the measurement circuitry the neural compound action potential response evoked by the neural stimulus;
derive, from the measured evoked response and the value (I) of the stimulus intensity, a feedback variable that is a proxy for a level of recruitment of neurons on the neural pathway by the neural stimulus, independent of the distance between the plurality of electrodes and the neural pathway; and
complete a feedback loop by using the feedback variable to control the stimulus intensity.

9. The device of claim 8 wherein the control unit is configured to derive the feedback variable using a value (V) of measured amplitude of the evoked neural compound action potential response.

10. The device of claim 9 wherein the control unit is configured to derive the feedback variable using a parameter k reflecting both a recruitment parameter n and a measurement parameter m, where m is not equal to n.

11. The device of claim 10 wherein the value of the parameter k is selected based on the stimulation and recording configurations in use.

12. The device of claim 10 wherein the value of the parameter k is determined clinically using a recruitment datum.

13. The device of claim 12 wherein the recruitment datum comprises one or more of: the patient's perceptual threshold, the patient's discomfort threshold, coverage of a certain area or body part, a qualitative characteristic of the patient's perception of a stimulation, the patient's perception of optimal comfort, an electrophysiological measure, the onset of muscle response/twitching, and a measure of neural activity.

14. The device of claim 8 wherein the control unit is configured to complete the feedback loop by:
generating an error signal from the feedback variable relative to a setpoint, and determining the next stimulus intensity value from the error signal.
